(19) United States
(12) Patent Application Publication
(10) Pub. No.: US 2026/0345718 A1
(43) Pub. Date: Nov. 5, 2026

(54) COMPOSITIONS FOR PROTECTION OF GASTRIC MUCOSA AND PREVENTION OR TREATMENT OF GASTRIC ULCERS, COMPRISING WHEATGRASS EXTRACT

(71) Applicants: KOREA INSTITUTE OF SCIENCE AND TECHNOLOGY, Seoul (KR); Gyeonggido Business & Science Accelerator, Suwon-si (KR)

(72) Inventors: Gyu-Sang HONG, Seoul (KR); Chun Whan CHOI, Seoul (KR); Uhtaek OH, Yongin-si (KR)

(73) Assignees: KOREA INSTITUTE OF SCIENCE AND TECHNOLOGY, Seoul (KR); Gyeonggido Business & Science Accelerator, Suwon-si (KR)

(21) Appl. No.: 19/563,448

(22) Filed: Mar. 11, 2026

(30) Foreign Application Priority Data
Apr. 22, 2025 (KR) 10-2025-0052594

(51) Int. Cl. A61K 36/899; A61K 31/7048; A61P 1/04

(52) U.S. Cl. CPC A61K 36/899; A61K 31/7048; A61P 1/04; A61K 2236/333; A61K 2236/39; A61K 2236/51

(57) ABSTRACT

The present invention relates to a composition for protecting the gastric mucosa or a pharmaceutical composition for preventing or treating gastrointestinal diseases, comprising a wheatgrass extract. The present wheatgrass extract exhibits an effect of preventing, treating, and improving gastrointestinal diseases caused by damage to the gastric mucosa, such as gastric ulcers, through its effects of protecting gastric mucosal cells from damage and promoting gastric mucus secretion.

### CROSS-REFERENCE TO RELATED APPLICATION(S)

This application claims the benefit under 35 USC 119(a) of Korean Patent Application No. 10-2025-0052594, filed with the Korean Intellectual Property Office on Apr. 22, 2025, the disclosure of which is incorporated herein by reference in its entirety.

### TECHNICAL FIELD

The present invention relates to a composition for protecting the gastric mucosa and preventing, improving, or treating gastric ulcers, a disease resulting from gastric mucosal damage, comprising wheatgrass extract and active ingredients extracted therefrom.

### BACKGROUND ART

The gastric mucosa is a crucial structure protecting the interior of the stomach, composed of the mucous layer, epithelial layer, lamina propria, and muscularis mucosae. The surface of the gastric mucosa contains mucous cells secreting mucus and prostaglandins, along with gastric mucosal epithelial cells. Mucus reduces the interaction between gastric acid and the gastric wall. Prostaglandins inhibit gastric acid secretion and increase blood flow to the gastric mucosa, aiding regeneration and healing, thereby playing a crucial role in preventing gastritis and gastric ulcers. Additionally, the gastric mucosa contains components and structures designed to protect the gastric wall, including proteins and dextran. Furthermore, epithelial cells possess rapid regenerative capacity, playing a role in swiftly repairing damaged tissue.

Gastric ulcers and gastric mucosal damage can arise from various causes, including excessive gastric acid secretion, Helicobacter pylori infection, use of nonsteroidal anti-inflammatory drugs (NSAIDs), stress, smoking, and alcohol consumption. Gastric ulcers are a condition in which the gastric mucosa is damaged, leading to a defect in the mucosal layer and the formation of a deep ulcer. In severe cases, the ulcer can invade the submucosa and the muscularis mucosae. Initial damage to the gastric mucosa begins as superficial injury accompanied by a minor inflammatory response. However, with persistent irritation, tissue necrosis and fibrosis can occur, leading to ulcer formation.

Previously, proton pump inhibitors (PPIs), H2 receptor antagonists, mucosal protectants, and antacids were used for the prevention and treatment of gastric ulcers and gastric mucosal damage. However, long-term use of these drugs can lead to side effects such as drug resistance, rebound increase in gastric acid secretion, and alterations in intestinal microbiota, necessitating safer and more effective alternative treatments. For example, antacids, the longest-used treatment for gastrointestinal disorders, directly neutralize stomach acid to protect the stomach lining, providing the fastest symptom relief. However, they do not inhibit gastric acid secretion and can cause side effects such as constipation, diarrhea, and urinary tract stones.

Therefore, to mitigate such side effects and develop drugs safe for long-term use, active research is underway to develop gastric mucosal protectants and gastric ulcer treatments using natural product-derived components. This approach can provide treatment effects that are safer and more stable over the long term compared to existing treatments.

### DISCLOSURE

### Technical Problem

While conducting research on natural substances with low side effects and safe gastric mucosal protective effects, or for the prevention or treatment of diseases caused by gastric mucosal damage, the present inventors discovered that wheatgrass extract and a specific compound contained therein, schaftoside, could possess protective effects on gastric mucosal cells and thereby prevent or treat gastric ulcers, leading to the completion of this invention.

Therefore, an objective of the present invention is to provide a pharmaceutical composition for protecting the gastric mucosa that comprises wheatgrass extract.

Another objective of the present invention is to provide a composition for the prevention, improvement, and treatment of gastric ulcers containing wheatgrass extract.

### Technical Solution

To achieve the above objectives, the present invention provides a pharmaceutical composition for protecting the gastric mucosa containing wheatgrass extract.

To achieve another objective of the present invention, the present invention provides a composition for the prevention, improvement, and treatment of gastric ulcers containing wheatgrass extract.

The invention is described in detail below.

As one embodiment of the present invention, the invention relates to a pharmaceutical composition for protecting the gastric mucosa containing wheatgrass extract.

In the present invention, “wheatgrass” refers to the young sprouts of wheat (Triticum aestivum), primarily harvested within 7 to 10 days after seed germination. Wheatgrass is widely used as a health supplement, either consumed as juice or processed into powder form. It is known as a superfood due to its rich content of vitamins A, C, E, K, and B complex, as well as various minerals and amino acids.

The composition of the present invention comprises an extract derived from wheatgrass (hereinafter “extract derived from wheatgrass” also referred to as “wheatgrass extract”). The extract is not limited by solvent or extraction method and may be extracted using one or more solvents selected from the group consisting of water, organic solvents, and mixtures thereof, provided that the active components can be extracted from wheatgrass. More preferably, the extract may be obtained using water, C1-C4 lower alcohols, or mixtures thereof.

Although not limited, extraction is preferably carried out by adding the extraction solvent at 1 to 20 times the weight of the wheatgrass, and more preferably at 5 to 15 times. The extraction temperature may be from 10 to 100 °C, preferably 50 to 80 °C, and the extraction time may be from 1 to 60 hours, preferably 10 to 48 hours, but is not limited thereto.

Conventional extraction methods may be employed, including vacuum high-temperature extraction, hot water extraction, reflux extraction, hydrothermal extraction, cold maceration extraction, room temperature extraction, ultrasonic extraction, or steam extraction.

Furthermore, the extract of the present invention may comprise an organic solvent fraction fractionated from an extract prepared using water, C1-C4 lower alcohols, or mixtures thereof. The organic solvent used for fractionation is not limited but may be selected from hexane, chloroform, ethyl acetate, n-butanol, or combinations thereof.

The concentration of wheatgrass extract contained in the composition of the present invention may be 0.001 to 10% (mg/mL), preferably 0.05 to 5% (mg/mL), and most preferably 0.1 to 1% (mg/mL), but is not specifically limited to these concentrations.

The wheatgrass extract of the present invention may contain schaftoside as an active ingredient. Specifically, the wheatgrass extract may contain schaftoside represented by the following chemical structure.

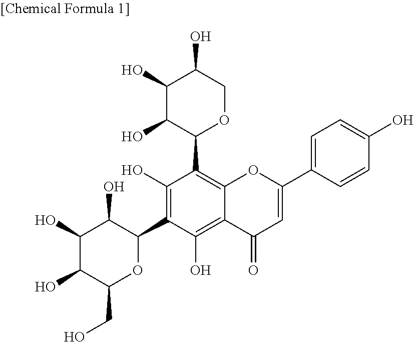 [SMILES: O=c1cc(-c2ccc(O)cc2)oc2c([C@@H]3OC[C@H](O)[C@H](O)[C@@H]3O)c(O)c([C@H]3O[C@@H](CO)[C@@H](O)[C@@H](O)[C@H]3O)c(O)c12]

The wheatgrass extract of the present invention exhibits a gastric mucosal protective effect. In the present invention, “gastric mucosal protection” refers to effects such as preventing damage to gastric mucosal cells and promoting mucus secretion, and the term “gastric mucosal damage” refers to injury to the gastric mucosa lining the inner surface of the stomach wall, which consists of simple columnar epithelium and can be caused by various factors that enhance gastric acid secretion, thereby leading to various gastrointestinal diseases.

In the present invention, gastrointestinal diseases may include acute gastritis, gastric ulcer, esophagitis, duodenitis, or duodenal ulcer. In particular, acute gastritis refers to acute inflammation of the stomach caused by gastric mucosal damage induced by nonsteroidal anti-inflammatory drugs (NSAIDs), steroidal anti-inflammatory drugs, medications, alcohol, food poisoning, food hypersensitivity, smoking, or stress.

The extract of the present invention may exert a gastric mucosal protective effect, meaning it prevents or protects against damage to the gastric mucosa or restores damaged gastric mucosa.

The mucosal protective effect of the composition containing the wheatgrass extract of the present invention on the digestive tract mucosa may be immediate or sustained. Specifically, it may exhibit an immediate mucosal protective effect when administered together with or immediately before factors irritating the gastric mucosa, or it may provide sustained protection and prevent gastrointestinal diseases through long-term, consistent dietary intake that strengthens the mucosa.

Furthermore, the composition containing the extract according to the present invention may provide protective effects against gastric mucosal damage caused by factors such as drugs that induce damage to the gastrointestinal mucosa, including alcohol and steroid preparations. Therefore, when administered concomitantly before or after the administration of alcohol or drugs, it can exhibit excellent preventive and therapeutic effects against gastric mucosal damage and the resulting gastrointestinal diseases.

The composition containing the extract according to the present invention can be utilized for various applications, such as pharmaceuticals, health functional foods, and functional foods.

The composition of the present invention may be a pharmaceutical composition for the prevention or treatment of gastrointestinal diseases, particularly acute gastritis or gastric ulcers.

The term “prevention” in this specification means any act that can suppress a disease or delay its onset through the administration of the pharmaceutical composition according to the present invention.

The term “treatment” in this specification refers to any act whereby symptoms are improved or alleviated by the administration of the pharmaceutical composition according to the present invention.

The pharmaceutical composition of the present invention may be formulated, according to conventional methods, into oral dosage forms such as powders, granules, tablets, capsules, suspensions, emulsions, syrups, and aerosols, as well as external preparations, suppositories, and sterile injectable solutions, and may additionally include carriers or excipients required for such formulation. Pharmaceutically acceptable carriers, excipients, and diluents that may be additionally included with the active ingredient include lactose, dextrose, sucrose, sorbitol, mannitol, xylitol, erythritol, maltitol, starch, acacia gum, alginate, gelatin, calcium phosphate, calcium silicate, cellulose, methylcellulose, microcrystalline cellulose, polyvinylpyrrolidone, water, methylparaben, propylparaben, magnesium stearate, and mineral oil. When formulating, commonly used diluents or excipients, such as fillers, extenders, binders, humectants, disintegrants, and surfactants, may be employed.

Solid dosage forms for oral administration include tablets, pills, powders, granules, and capsules, and these solid dosage forms may be prepared by mixing the extract or compound with at least one excipient such as starch, calcium carbonate, sucrose or lactose, and gelatin. In addition to such simple excipients, lubricants such as magnesium stearate and talc may also be used. Liquid formulations for oral administration include suspensions, solutions, emulsions, and syrups, and in addition to commonly used simple diluents such as water and liquid paraffin, various excipients, for example humectants, sweeteners, flavorings, and preservatives, may be included.

Formulations for parenteral administration include sterile aqueous solutions, non-aqueous solvents, suspensions, emulsions, freeze-dried preparations, and suppositories. Non-aqueous solvents and suspensions may employ propylene glycol, polyethylene glycol, vegetable oils such as olive oil, or injectable esters such as ethyl oleate. Bases for suppositories may include witepsol, macrogol, Tween 61, cocoa butter, lauric fat, and glycerogelatin.

The pharmaceutical composition of the present invention may be administered orally or parenterally (for example, by intravenous, subcutaneous, intraperitoneal, or topical administration), depending on the intended use. The dosage may vary depending on the patient's condition and body weight, the severity of the disease, the dosage form, the route of administration, and the time of administration, and may be appropriately selected by those skilled in the art.

The pharmaceutical composition of the present invention is administered in a pharmaceutically effective amount. In the present invention, the term “pharmaceutically effective amount” refers to a reasonable amount applicable to medical treatment and sufficient to treat the disease, and the criteria therefor may be determined in consideration of factors such as the patient's disease, severity, drug activity, sensitivity to the drug, time and route of administration, rate of excretion, duration of treatment, concomitant components, and other factors. The pharmaceutical composition of the present invention may be administered as a single therapeutic agent or in combination with other therapeutic agents, and may be administered sequentially or simultaneously with conventional therapeutic agents. In view of all of the above factors, the dosage may be determined at a level that minimizes side effects, and such dosage can be readily determined by those skilled in the art. Specifically, the dosage of the pharmaceutical composition may vary depending on the patient's age, body weight, severity, sex, and the like; in general, it may be administered in an amount of 0.001 to 1000 mg per kg of body weight, preferably 0.01 to 500 mg per kg, and more preferably 10 to 300 mg per kg, once to three times per day, daily or every other day. However, this is merely exemplary, and the dosage may be adjusted as needed.

In addition, the composition of the present invention may be a food or a health functional food. In particular, the term “health functional food” refers to a food manufactured and processed using raw materials or ingredients having functionality beneficial to the human body, as defined in the Korean Health Functional Food Act, and the term “functionality” means that the food is ingested for the purpose of obtaining effects useful for health, such as regulating nutrients or exerting physiological actions, with respect to the structure and functions of the human body.

The food or health functional food of the present invention may be manufactured and processed into pharmaceutical dosage forms such as powders, granules, tablets, capsules, pills, suspensions, emulsions, and syrups, or into health functional foods such as tea bags, infusions, beverages, candies, jellies, and gums for the purpose of preventing and improving gastrointestinal diseases or protecting the gastric mucosa.

The food or health functional food composition of the present invention may be used as a food additive and may be commercialized alone or in combination with other ingredients. In addition, it may further include nutritional supplements, vitamins, electrolytes, flavorings, colorants and flavor enhancers, pectic acid and salts thereof, alginic acid and salts thereof, organic acids, protective colloidal thickeners, pH adjusters, stabilizers, preservatives, glycerin, alcohols, and carbonating agents used in carbonated beverages. These components may be used alone or in combination, and may be combined and used at appropriate concentrations.

### Advantageous Effects

The present invention relates to a composition for protecting the gastric mucosa, or for preventing, improving, or treating gastrointestinal diseases, comprising wheatgrass extract. The composition provides protective effects for gastric mucosal cells and alleviates diseases caused by mucosal damage. It can be used as a composition for pharmaceuticals, health functional foods, functional foods, or animal feed additives.

### DESCRIPTION OF DRAWINGS

FIG. 1 is a diagram illustrating the process of extracting and fractionating the active components, including schaftoside, from wheatgrass extract.

FIG. 2 shows the HPLC (High-Performance Liquid Chromatography) analysis results for the substances schaftoside and isoorientin identified in the wheatgrass extract of the present invention.

FIGS. 3A to 3G illustrate the experimental process and results for alleviating gastric ulcers and protecting the gastric mucosa in mice using wheatgrass ethanol extract and its butanol fraction. FIG. 3A is a schematic of the administered extract, dosage, and administration schedule. FIG. 3B shows images confirming the administration results on gastric tissue from mice with induced gastric ulcers, and FIG. 3C presents the quantitative analysis results. FIG. 3D shows the H&E staining results of the tissue with induced gastric ulcers, and FIG. 3E presents the quantitative analysis results. FIG. 3F shows the PAS staining results of the gastric mucosal tissue, and FIG. 3G is a diagram confirming the change in gastric mucosal thickness.

FIGS. 4A to 4G confirm the ulcer-alleviating and preventive effects via gastric mucosal barrier protection when schaftoside was administered to the gastric ulcer-induced mouse model. Each diagram is as follows: (A) Overview of the schaftoside administration experiment in the HCl/EtOH-induced gastric ulcer mouse model, (B) macroscopic image of the cross-section of the stomach where the ulcer was induced, (C) gastric lesion index (%) after oral administration (5 days): non-induced gastric ulcer group (Control), 0.5% Na-CMC solution (control group), schaftoside (60 mg/kg/day or 80 mg/kg/day), or schaftoside (80 mg/kg/day) co-administered with the TRPM5 inhibitors Flufenamic Acid (FFA) and Triphenylphosphine oxide (TTPO), (E) Representative images of H&E-stained gastric tissue from each experimental group. Scale bar: 50 μm, (E) Blank index via H&E staining, (F) Gastric mucosa visualization via PAS staining, (G) Mucosal layer thickness (μm) measurements. Numbers in parentheses indicate the number of analyzed tissue sections (3 sections per mouse).

FIGS. 5A to 5E show the results confirming the calcium response and gastric mucus secretion effects of schaftoside (SCH) on gastric mucosal cells (AGS). The images are as follows: (A) AB/PAS-stained AGS cell image, (B) color intensity evaluation results from panel B, (C) Fluorescence signal representing the average F/F0 ratio of AGS cells after SCH treatment, (E) Area under the curve (AUC) in panel E, (D) Fluorescence signal representing the average F/F0 ratio of AGS cells in panel G, (E) Maximum inhibition rate (%) of cells activated by TPPO (50 μM) treatment (F/F0).

### MODE OF THE INVENTION

Hereinafter, the present specification is described in detail with reference to examples for the purpose of providing a more complete explanation. However, the examples described herein may be modified in various forms, and the scope of the present specification is not intended to be limited to the examples described below. The examples provided herein are intended to provide a more complete explanation of the present specification to those skilled in the art.

### Example 1. Preparation of Wheatgrass Extract and Isolation of Active Components

### 1-1. Preparation of the Extract

Wheatgrass (1 kg) was cold-soaked in 70% ethanol (5 L), filtered, and the filtrate was concentrated under reduced pressure to yield 130 g of extract.

The extract was suspended in purified water, transferred to a separatory funnel, and mixed thoroughly with an equal volume of water-saturated butanol (n-butanol). The mixture was allowed to settle, and the aqueous layer and n-butanol layer were separated. This water-saturated butanol extraction process was repeated 2 to 3 times. The recovered butanol layer was dehydrated, filtered, and the filtrate was concentrated under reduced pressure to obtain the butanol fraction.

### 1-2. Isolation of Schaftoside and Isoorientin

The active components contained in the wheatgrass extract obtained in Section 1-1 were analyzed and identified by HPLC (high-performance liquid chromatography). As a result, schaftoside and isoorientin were confirmed to be present, as shown in FIG. 2, and, in order to isolate these components, additional fractionation using organic solvents was carried out.

Specifically, 100 g of 70% ethanol extract of wheatgrass was suspended in 1 L of 10% methanol, passed through Diaion HP-20 resin (1 kg), eluted with 10% methanol solvent, and then subjected to a solvent gradient up to 80% methanol to divide it into 5 fractions. Each fraction was concentrated under reduced pressure. Fractions 2 and 4 were separated and purified for schaftoside and isoorientin via preparative HPLC (mobile phase: water/methanol 90-20%, flow rate 5 mL/min).

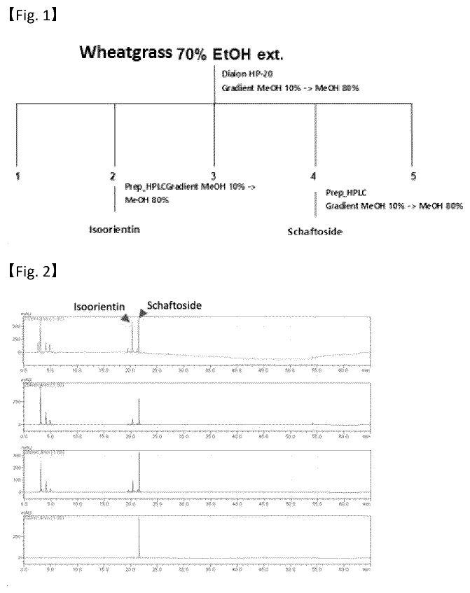

The above isolation process is illustrated in FIG. 1.

### Example 2. Effects of Administering Wheatgrass Extract

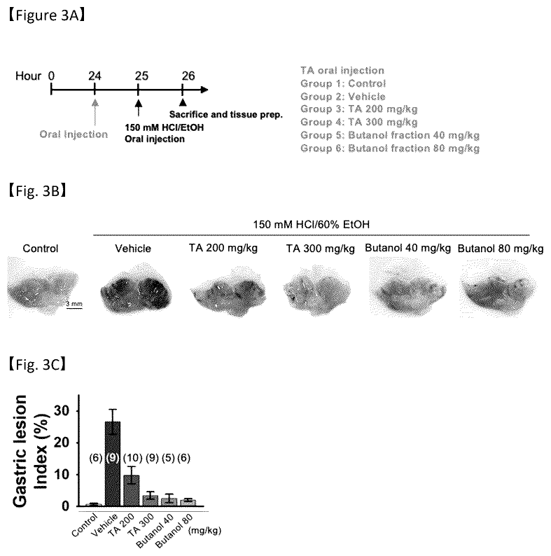

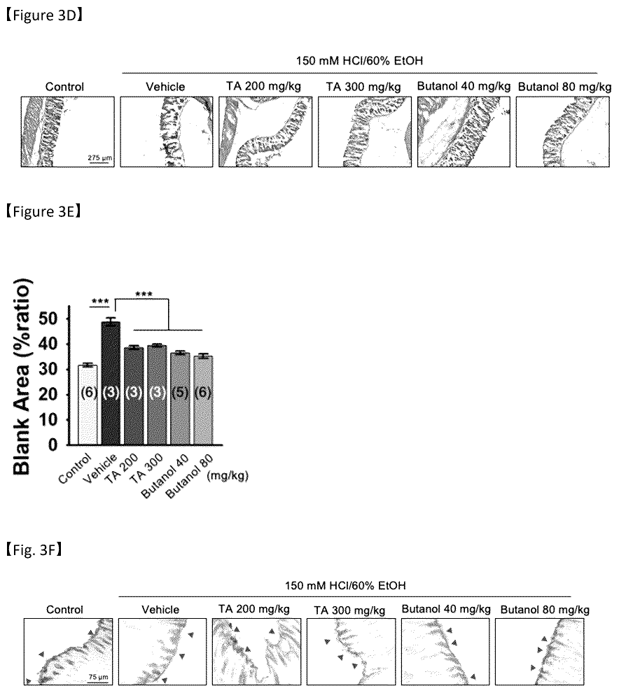

Mice (C57BL/6) were orally administered ethanol extract of wheatgrass (TA) at 200 to 300 mg/kg, and the butanol fraction of the wheatgrass extract (TA) at 40 mg/kg and 80 mg/kg, respectively. One hour after administration, gastric ulcers were induced by administering 150 mM HCl/60% ethanol. After an additional 1 hour, the mice were sacrificed to evaluate the ulcer-healing effect on the gastric wall (FIG. 3A). Compared to the control group (vehicle, Veh), ulceration of the gastric wall was significantly reduced in the groups administered wheatgrass extract and its butanol fraction, as shown in FIG. 3B, which was also supported by quantitative analysis results (FIG. 3C). Furthermore, H&E staining of the tissue (FIG. 3D) and quantitative analysis using the lesion index (FIG. 3E) also confirmed a marked reduction in gastric ulcers.

Furthermore, to confirm the gastric mucus secretion-promoting effect of wheatgrass extract and its butanol fraction, PAS staining was performed on gastric mucosal cells. As shown in FIG. 3F, PAS staining intensity was significantly increased in all treatment groups, and an increase in gastric mucosal thickness was observed in all groups (FIG. 3G).

### Example 3. Effects of Administration of Schaftoside

### 3-1. Gastric Ulcer Alleviation Effect of Administration of Schaftoside

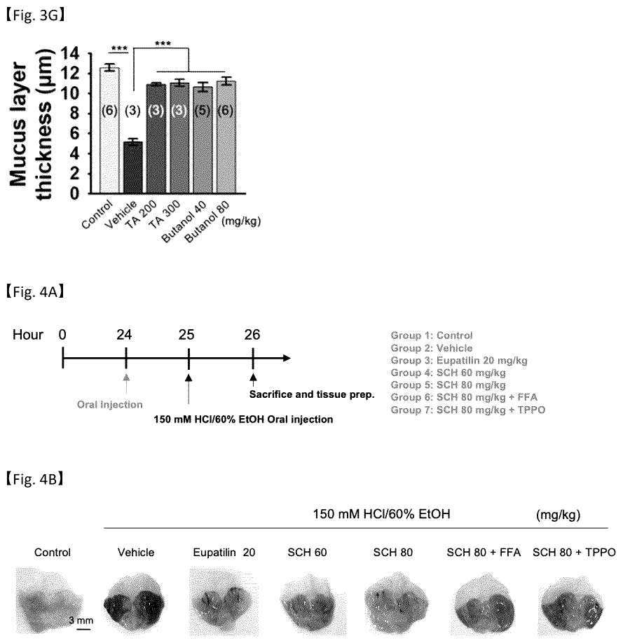

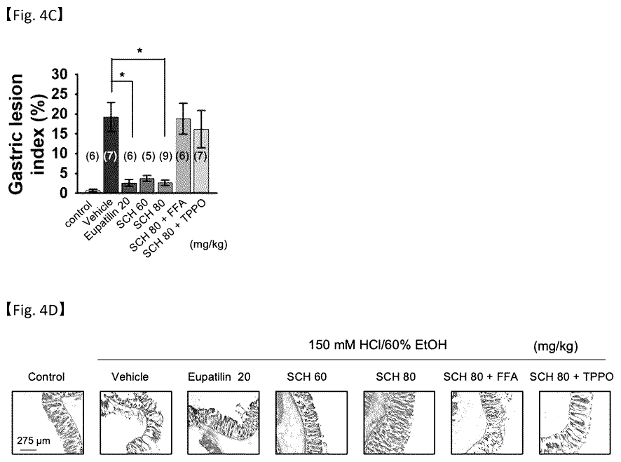

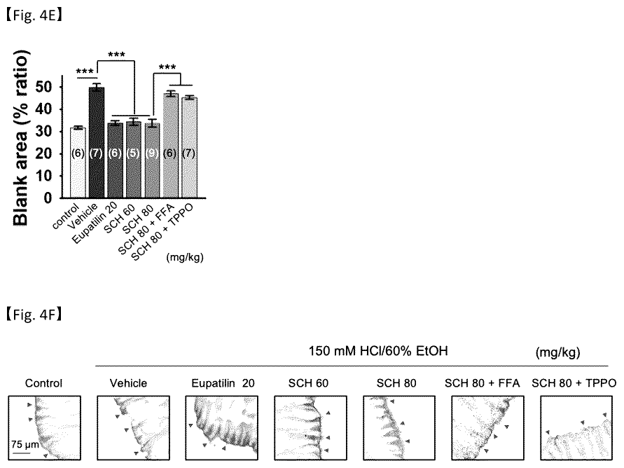

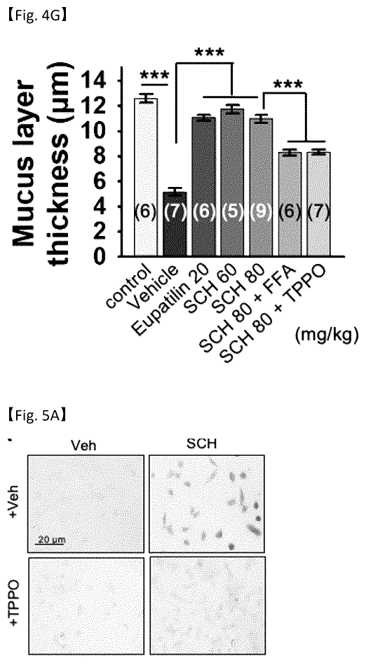

Experiments were conducted to evaluate the ameliorative effect of schaftoside on HCl/EtOH-induced gastric ulcers. Mice received a single oral dose of schaftoside (60 and 80 mg/kg; groups 3 and 4), and, after schaftoside administration, the TRPM5 inhibitors FFA and TPPO were administered orally to confirm the schaftoside-mediated effect (groups 5 and 6). Gastric ulcers were induced in each treatment group by administering 150 mM HCl/60% ethanol 1 hour after drug administration, and 1 hour later the mice were sacrificed to assess the ulcer-alleviating effect on the gastric wall (FIG. 4A).

As shown in FIG. 4B, the mean gastric lesion area progressively decreased in the schaftoside-treated groups, with a particularly marked reduction observed in the 80 mg/kg group (2.68±0.74%) compared with the vehicle-treated group (25.99±2.99%). However, intraperitoneal administration of the TRPM5 inhibitors FFA and TPPO abolished the effects of oral schaftoside at 60 and 80 mg/kg. This effect was observed at a level similar to that of the positive control eupatilin (EUP), and this was further confirmed by the quantitative analysis results shown in FIG. 4C. In addition, H&E staining of the gastric tissue (FIG. 4D) and quantitative analysis using a lesion (blank) index (FIG. 4E) also demonstrated a significant reduction in gastric ulcers in the schaftoside-treated groups.

Furthermore, visualization of the gastric mucosa by PAS staining revealed that the mucosal layer thickness (12.80±0.57 μm) was significantly increased in the schaftoside-treated group (80 mg/kg) compared with the vehicle-treated group (7.12±0.40 μm) (FIG. 4F and FIG. 4G).

These results indicate that schaftoside rapidly promotes mucus secretion and exerts a gastric mucosal protective effect. Accordingly, in the HCl/EtOH-induced gastric ulcer mouse model, schaftoside is suggested to act as a key therapeutic candidate that provides gastric protection by increasing mucus production and secretion.

### 3-2. Gastric Mucus Secretion Promoting Effect of Administration of Schaftoside

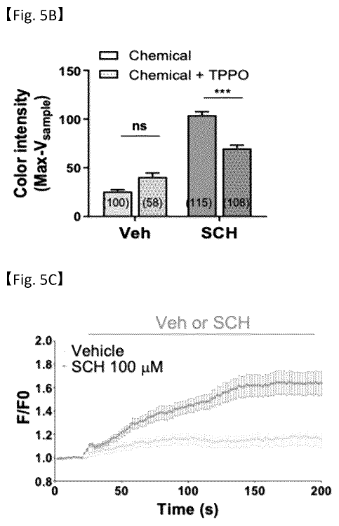

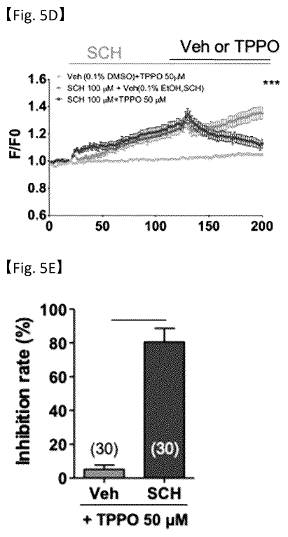

To confirm the gastric mucus secretion-promoting effect of schaftoside, PAS staining was performed on gastric mucosal cells (AGS), as shown in FIGS. 5A and 5B. PAS signal intensity (104±3.45) was significantly increased in the schaftoside-treated group, whereas the PAS signal (69.94±3.22) was markedly reduced upon treatment with the TRPM5 protein inhibitor TPPO, suggesting that mucus production induced by schaftoside is mediated by TRPM5 activation.

To investigate whether schaftoside affects intracellular Ca²⁺ signaling, Ca²⁺ imaging experiments were performed in AGS cells, and a significant increase in intracellular calcium concentration [Ca²⁺]ᵢ was observed in the schaftoside (100 μM)-treated group (FIGS. 5C and 5D).

Furthermore, to determine whether the Ca²⁺ signaling induced by schaftoside is mediated via the TRPM5-NCX pathway, additional Ca²⁺ imaging experiments were conducted. The results showed a marked increase in [Ca^{2*}]ᵢ in the schaftoside-treated group, and this increase was significantly suppressed to approximately 80% by pretreatment with TPPO (50 μM) (FIGS. 5E and 5F).

These findings demonstrate that schaftoside activates the TRPM5-NCX pathway to promote Ca²⁺ influx, ultimately enhancing mucus (mucin) production in AGS cells, and thus indicate that schaftoside has therapeutic potential for promoting the defensive mechanisms of the gastric mucosa.

Thus far, the present invention has been described with reference to its preferred embodiments. Those skilled in the art to which the present invention pertains will understand that the invention may be embodied in modified forms without departing from its essential character. Therefore, the disclosed embodiments should be considered from an illustrative perspective rather than a limiting one. The scope of the present invention is defined not by the foregoing description but by the appended claims, and all differences within the scope of the claims should be interpreted as falling within the scope of the present invention.

1. A composition for protecting the gastric mucosa, comprising wheatgrass extract.

2. The composition of claim 1, wherein the wheatgrass extract is extracted using water, a C1-C4 lower alcohol, or a mixture thereof as the extraction solvent.

3. The composition of claim 1, wherein the wheatgrass extract comprises an organic solvent fraction selected from hexane, chloroform, ethyl acetate, n-butanol, or combinations thereof, which is fractionated from the extract obtained using water, a C1-C4 lower alcohol, or a mixture thereof as the extraction solvent.

4. The composition of claim 1, wherein the composition prevents damage to gastric mucosal cells or promotes mucus secretion of gastric mucosal cells.

5. The composition of claim 1, wherein the composition comprises schaftoside as an active ingredient.

6. The composition of claim 1, wherein the wheatgrass extract is administered at a dosage of 0.001 to 1000 mg per kg of body weight.

7. A health functional food comprising the composition according to claim 1.

8. A pharmaceutical composition for the prevention or treatment of gastrointestinal diseases containing wheatgrass extract.

9. The composition of claim 8, wherein the wheatgrass extract is extracted using water, C1-C4 lower alcohols, or mixtures thereof as the extraction solvent.

10. The composition of claim 8, wherein the wheatgrass extract comprises an organic solvent fraction selected from hexane, chloroform, ethyl acetate, n-butanol, or combinations thereof, which is fractionated from the extract obtained using water, a C1-C4 lower alcohol, or a mixture thereof as the extraction solvent.

11. The composition of claim 8, wherein the composition comprises schaftoside as an active ingredient.

12. The composition of claim 8, wherein the gastrointestinal disease is one or more of gastritis, gastric ulcer, esophagitis, duodenitis, or duodenal ulcer occurring in the upper gastrointestinal tract.

13. A method for treating a gastrointestinal disease in a subject in need thereof, comprising administering to the subject a therapeutically effective amount of the wheatgrass extract of claim 1 or schaftoside isolated therefrom.
